(19) United States
(12) Patent Application Publication
(10) Pub. No.: US 2026/0234291 A1
(43) Pub. Date: Aug. 13, 2026

(54) MUTATED IMMUNOGLOBULIN-BINDING POLYPEPTIDES

(71) Applicant: Cytiva Bioprocess R&D AB, Uppsala (SE)

(72) Inventors: Gustav RODRIGO, Uppsala (SE); Tomas Bjorkman, Uppsala (SE); Mats Ander, Uppsala (SE)

(21) Appl. No.: 19/569,673

(22) Filed: Mar. 17, 2026

Related U.S. Application Data
(63) Continuation of application No. 19/370,592, filed on Oct. 27, 2025.
Application No. 19/370,592 is a continuation of application No. 18/093,624, filed on Jan. 5, 2023, now abandoned.
Application No. 18/093,624 is a continuation of application No. 16/594,731, filed on Oct. 7, 2019, now abandoned.
Application No. 16/594,731 is a continuation of application No. 15/526,941, filed on Nov. 16, 2015, now Pat. No. 10,501,557.

(30) Foreign Application Priority Data
Nov. 17, 2014 (SE) 1451376-6
Jun. 12, 2015 (GB) 1510261.9
Jun. 12, 2015 (GB) 1510263.5

(51) Int. Cl. C07K 16/46; B01D 15/38; B01J 20/289; B01J 20/32; C07K 1/22; C07K 14/31; C07K 16/00; C07K 16/06

(52) U.S. Cl. CPC C07K 16/46; B01D 15/3809; B01J 20/289; B01J 20/3219; B01J 20/3274; C07K 1/22; C07K 14/31; C07K 16/00; C07K 16/065

(57) ABSTRACT

An Fc-binding polypeptide of improved alkali stability, comprising a mutant of an Fc-binding domain of Staphylococcus Protein A (SpA), as defined by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 22, SEQ ID NO 51 or SEQ ID NO 52 wherein at least the asparagine or serine residue at the position corresponding to position 11 in SEQ ID NO: 4-7 has been mutated to an amino acid selected from the group consisting of glutamic acid, lysine, tyrosine, threonine, phenylalanine, leucine, isoleucine, tryptophan, methionine, valine, alanine, histidine and arginine.

### CROSS-REFERENCE TO RELATED APPLICATIONS

This application is a continuation of U.S. patent application Ser. No. 19/370,592, filed Oct. 27, 2025, which is a continuation of U.S. patent application Ser. No. 18/093,624, filed Jan. 5, 2023, which is a continuation of U.S. patent application Ser. No. 16/594,731, filed Oct. 7, 2019, which is a continuation of U.S. patent application Ser. No. 15/526,941, filed May 15, 2017, now U.S. Pat. No. 10,501,557, issued Dec. 10, 2019, which is a filing under 35 U.S.C. 371 of International Application No. PCT/EP2015/076639, filed Nov. 16, 2015, which claims priority to application no. 1510263.5 filed in Great Britain on Jun. 12, 2015, application no. 1510261.9 filed in Great Britain on Jun. 12, 2015, and application no. 1451376-6 filed in Sweden on Nov. 17, 2014, the entire disclosures of each of which are hereby incorporated by reference.

### SEQUENCE LISTING

The instant application contains a Sequence Listing which has been submitted electronically in XML format and is hereby incorporated by reference in its entirety. Said XML copy, created on Mar. 16, 2026, is named 100001_00175_Sequence_Listing.xml and is 57,047 bytes in size.

### TECHNICAL FIELD OF THE INVENTION

The present invention relates to the field of affinity chromatography, and more specifically to mutated immunoglobulin-binding domains of Protein A, which are useful in affinity chromatography of immunoglobulins. The invention also relates to multimers of the mutated domains and to separation matrices containing the mutated domains or multimers.

### BACKGROUND OF THE INVENTION

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their respective mAb manufacturing processes whilst controlling the associated costs.

Affinity chromatography is used in most cases, as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as but not limited to serum or plasma preparations or cell culture derived feed stocks. An example of such a protein is staphylococcal protein A, containing domains capable of binding to the Fc and Fab portions of IgG immunoglobulins from different species. These domains are commonly denoted as the E-, D-, A-, B- and C-domains.

Staphylococcal protein A (SpA) based reagents have due to their high affinity and selectivity found a widespread use in the field of biotechnology, e.g. in affinity chromatography for capture and purification of antibodies as well as for detection or quantification. At present, SpA-based affinity medium probably is the most widely used affinity medium for isolation of monoclonal antibodies and their fragments from different samples including industrial cell culture supernatants. Accordingly, various matrices comprising protein A-ligands are commercially available, for example, in the form of native protein A (e.g. Protein A SEPHAROSE™, GE Healthcare, Uppsala, Sweden) and also comprised of recombinant protein A (e.g. rProtein A-SEPHAROSE™, GE Healthcare). More specifically, the genetic manipulation performed in the commercial recombinant protein A product is aimed at facilitating the attachment thereof to a support and at increasing the productivity of the ligand.

These applications, like other affinity chromatography applications, require comprehensive attention to definite removal of contaminants. Such contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such contaminants from the matrix is usually performed after a first elution of the desired product in order to regenerate the matrix before subsequent use. Such removal usually involves a procedure known as cleaning-in-place (CIP), wherein agents capable of eluting contaminants from the stationary phase are used. One such class of agents often used is alkaline solutions that are passed over said stationary phase. At present the most extensively used cleaning and sanitizing agent is NaOH, and the concentration thereof can range from 0.1 up to e.g. 1 M, depending on the degree and nature of contamination. This strategy is associated with exposing the matrix to solutions with pH-values above 13. For many affinity chromatography matrices containing proteinaceous affinity ligands such alkaline environment is a very harsh condition and consequently results in decreased capacities owing to instability of the ligand to the high pH involved.

An extensive research has therefore been focused on the development of engineered protein ligands that exhibit an improved capacity to withstand alkaline pH-values. For example, Gülich et al. (Susanne Gülich, Martin Linhult, Per-Åke Nygren, Mathias Uhlén, Sophia Hober, Journal of Biotechnology 80 (2000), 169-178) suggested protein engineering to improve the stability properties of a Streptococcal albumin-binding domain (ABD) in alkaline environments. Gülich et al. created a mutant of ABD, wherein all the four asparagine residues have been replaced by leucine (one residue), aspartate (two residues) and lysine (one residue). Further, Gülich et al. report that their mutant exhibits a target protein binding behavior similar to that of the native protein, and that affinity columns containing the engineered ligand show higher binding capacities after repeated exposure to alkaline conditions than columns prepared using the parental non-engineered ligand. Thus, it is concluded therein that all four asparagine residues can be replaced without any significant effect on structure and function.

Recent work shows that changes can also be made to protein A (SpA) to effect similar properties. US patent application publication US 2005/0143566, which is hereby incorporated by reference in its entirety, discloses that when at least one asparagine residue is mutated to an amino acid other than glutamine or aspartic acid, the mutation confers an increased chemical stability at pH-values of up to about 13-14 compared to the parental SpA, such as the B-domain of SpA, or Protein Z, a synthetic construct derived from the B-domain of SpA (U.S. Pat. No. 5,143,844, incorporated by reference in its entirety). The authors show that when these mutated proteins are used as affinity ligands, the separation media as expected can better withstand cleaning procedures using alkaline agents. Further mutations of protein A domains with the purpose of increasing the alkali stability have also been published in WO 2008/039141, JP 2006304633A, EP 1992692A1, EP 2202310A2, WO 2010/110288, WO 2012/086660, WO 2012/083425, WO 2012/087230 and WO 2014/146350, all of which are hereby incorporated by reference in their entireties. However, the currently available mutants are still sensitive to alkaline pH and the NaOH concentration during cleaning is usually limited to 0.1 M, which means that complete cleaning is difficult to achieve. Higher NaOH concentrations, which would improve the cleaning, lead to unacceptable capacity losses.

There is thus still a need in this field to obtain a separation matrix containing protein ligands having a further improved stability towards alkaline cleaning procedures.

### SUMMARY OF THE INVENTION

One aspect of the invention is to provide a polypeptide with improved alkaline stability. This is achieved with a polypeptide as defined in claim 1.

One advantage is that the alkaline stability is improved over the parental polypeptides, with a maintained highly selective binding towards immunoglobulins and other Fc-containing proteins.

A second aspect of the invention is to provide a multimer with improved alkaline stability, comprising a plurality of polypeptides. This is achieved with a multimer as defined in the claims.

A third aspect of the invention is to provide a nucleic acid or a vector encoding a polypeptide or multimer with improved alkaline stability. This is achieved with a nucleic acid or vector as defined in the claims.

A fourth aspect of the invention is to provide an expression system capable of expressing a polypeptide or multimer with improved alkaline stability. This is achieved with an expression system as defined in the claims.

A fifth aspect of the invention is to provide a separation matrix capable of selectively binding immunoglobulins and other Fc-containing proteins and exhibiting an improved alkaline stability. This is achieved with a separation matrix as defined in the claims.

A sixth aspect of the invention is to provide an efficient and economical method of isolating an immunoglobulin or other Fc-containing protein. This is achieved with a method as defined in the claims.

Further suitable embodiments of the invention are described in the dependent claims.

### Definitions

The terms “antibody” and “immunoglobulin” are used interchangeably herein, and are understood to include also fragments of antibodies, fusion proteins comprising antibodies or antibody fragments and conjugates comprising antibodies or antibody fragments.

The terms an “Fc-binding polypeptide” and “Fc-binding protein” mean a polypeptide or protein respectively, capable of binding to the crystallisable part (Fc) of an antibody and includes e.g. Protein A and Protein G, or any fragment or fusion protein thereof that has maintained said binding property.

The term “linker” herein means an element linking two polypeptide units, monomers or domains to each other in a multimer.

The term “spacer” herein means an element connecting a polypeptide or a polypeptide multimer to a support.

### BRIEF DESCRIPTION OF FIGURES

FIG. 1 shows an alignment of the Fc-binding domains as defined by SEQ ID NO:1-7 and 51-52.

FIG. 2 shows results from Example 2 for the alkali stability of parental and mutated tetrameric Zvar (SEQ ID NO 7) polypeptide variants coupled to an SPR biosensor chip.

FIG. 3 shows results from Example 4 for the alkali stability (0.5 M NaOH) of parental and mutated tetrameric Zvar (SEQ ID NO 7) polypeptide variants coupled to agarose beads.

FIG. 4 shows results from Example 4 for the alkali stability (1.0 M NaOH) of parental and mutated tetrameric Zvar (SEQ ID NO 7) polypeptide variants coupled to agarose beads.

### DETAILED DESCRIPTION OF EMBODIMENTS

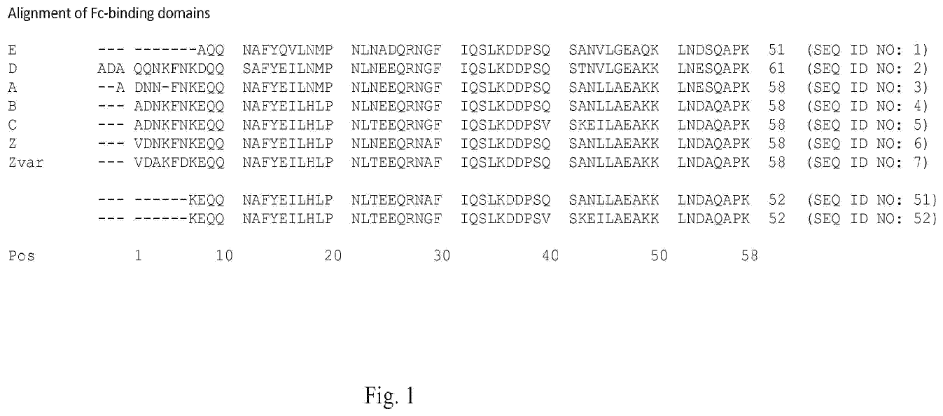

In one aspect the present invention discloses an Fc-binding polypeptide, which comprises, or consists essentially of, a mutant of an Fc-binding domain of Staphylococcus Protein A (SpA), as defined by, or having at least 90%, at least 95% or at least 98% identity to, SEQ ID NO: 1 (E-domain), SEQ ID NO: 2 (D-domain), SEQ ID NO:3 (A-domain), SEQ ID NO: 22 (variant A-domain), SEQ ID NO: 4 (B-domain), SEQ ID NO: 5 (C-domain), SEQ ID NO: 6 (Protein Z), SEQ ID NO:7 (Zvar), SEQ ID NO 51 (Zvar without the linker region amino acids 1-6) or SEQ ID NO 52 (C-domain without the linker region amino acids 1-6) as illustrated in FIG. 1, wherein at least the asparagine (or serine, in the case of SEQ ID NO 2) residue at the position* corresponding to position 11 in SEQ ID NO:4-7 has been mutated to an amino acid selected from the group consisting of glutamic acid, lysine, tyrosine, threonine, phenylalanine, leucine, isoleucine, tryptophan, methionine, valine, alanine, histidine and arginine. Protein Z (SEQ ID NO:6) is a mutated B-domain as disclosed in U.S. Pat. No. 5,143,844, while SEQ ID NO 7 denotes a further mutated variant of Protein Z, here called Zvar, with the mutations N3A,N6D,N23T. SEQ ID NO:22 is a natural variant of the A-domain in Protein A from Staphylococcus aureus strain N315, having an A46S mutation, using the position terminology of FIG. 1. The mutation of N11 in these domains confers an improved alkali stability in comparison with the parental domain/polypeptide, without impairing the immunoglobulin-binding properties. Hence, the polypeptide can also be described as an Fc- or immunoglobulin-binding polypeptide, or alternatively as an Fc- or immunoglobulin-binding polypeptide unit. *Throughout this description, the amino acid residue position numbering convention of FIG. 1 is used, and the position numbers are designated as corresponding to those in SEQ ID NO 4-7.

In alternative language, the invention discloses an Fc-binding polypeptide which comprises a sequence as defined by, or having at least 90%, at least 95% or at least 98% identity to SEQ ID NO 53.

SEQ ID NO 53
KEX₁Q X₂AFYEILX₃LP NLTEEQRX₄X₅F IX₆X₇LKDX₈PSX₉
SX₁₀X₁₁X₁₂LAEAKX₁₃ X₁₄NDAQAPK

where individually of each other:
X₁=A or Q
X₂=E,K,Y,T,F,L,W,I,M,V,A,H or R
X₃=H or K
X₄=A or N
X₅=A or G
X₆=Q or E
X₇=S or K
X₈=E or D
X₉=Q or V
X₁₀=K,R or A
X₁₁=A,E or N
X₁₂=I or L
X₁₃=K or R
X₁₄=L or Y

The N11 (X₂) mutation (e.g. a N11E or N11K mutation) may be the only mutation or the polypeptide may also comprise further mutations, such as substitutions in at least one of the positions corresponding to positions 3, 6, 9, 10, 15, 18, 23, 28, 29, 32, 33, 36, 37, 40, 42, 43, 44, 47, 50, 51, 55 and 57 in SEQ ID NO:4-7. In one or more of these positions, the original amino acid residue may e.g. be substituted with an amino acid which is not asparagine, proline or cysteine. The original amino acid residue may e.g. be substituted with an alanine, a valine, a threonine, a serine, a lysine, a glutamic acid or an aspartic acid. Further, one or more amino acid residues may be deleted, e.g. from positions 1-6 and/or from positions 56-58.

In some embodiments, the amino acid residue at the position corresponding to position 9 in SEQ ID NO:4-7 (X₁) is an amino acid other than glutamine, asparagine, proline or cysteine, such as an alanine. The combination of the mutations at positions 9 and 11 provides particularly good alkali stability, as shown by the examples. In specific embodiments, in SEQ ID NO: 7 the amino acid residue at position 9 is an alanine and the amino acid residue at position 11 is a lysine or glutamic acid, such as a lysine. Mutations at position 9 are also discussed in copending application PCT/SE2014/050872, which is hereby incorporated by reference in its entirety.

In some embodiments, the amino acid residue at the position corresponding to position 50 in SEQ ID NO:4-7 (X₁₃) is an arginine or a glutamic acid.

In certain embodiments, the amino acid residue at the position corresponding to position 3 in SEQ ID NO:4-7 is an alanine and/or the amino acid residue at the position corresponding to position 6 in SEQ ID NO:4-7 is an aspartic acid. One of the amino acid residues at positions 3 and 6 may be an asparagine and in an alternative embodiment both amino acid residues at positions 3 and 6 may be asparagines.

In some embodiments the amino acid residue at the position corresponding to position 43 in SEQ ID NO:4-7 (X₁₁) is an alanine or a glutamic acid, such as an alanine. In specific embodiments, the amino acid residues at positions 9 and 11 in SEQ ID NO: 7 are alanine and lysine/glutamic acid respectively, while the amino acid residue at position 43 is alanine or glutamic acid.

In certain embodiments the amino acid residue residue at the position corresponding to position 28 in SEQ ID NO:4-7 (X₅) is an alanine or an asparagine, such as an alanine.

In some embodiments the amino acid residue at the position corresponding to position 40 in SEQ ID NO:4-7 (X₉) is selected from the group consisting of asparagine, alanine, glutamic acid and valine, or from the group consisting of glutamic acid and valine. In specific embodiments, the amino acid residues at positions 9 and 11 in SEQ ID NO: 7 are alanine and glutamic acid respectively, while the amino acid residue at position 40 is valine. Optionally, the amino acid residue at position 43 may then be alanine or glutamic acid.

In certain embodiments, the amino acid residue at the position corresponding to position 42 in SEQ ID NO:4-7 (X₁₀) is an alanine, lysine or arginine.

In some embodiments the amino acid residue at the position corresponding to position 18 in SEQ ID NO:4-7 (X₃) is a lysine or a histidine, such as a lysine.

In certain embodiments the amino acid residue at the position corresponding to position 33 in SEQ ID NO:4-7 (X₇) is a lysine or a serine, such as a lysine.

In some embodiments the amino acid residue at the position corresponding to position 37 in SEQ ID NO:4-7 (X₈) is a glutamic acid or an aspartic acid, such as a glutamic acid.

In certain embodiments the amino acid residue at the position corresponding to position 51 in SEQ ID NO:4-7 (X₁₄) is a tyrosine or a leucine, such as a tyrosine.

In some embodiments, the amino acid residue at the position corresponding to position 44 in SEQ ID NO:4-7 (X₁₂) is a leucine or an isoleucine. In specific embodiments, the amino acid residues at positions 9 and 11 in SEQ ID NO: 7 are alanine and lysine/glutamic acid respectively, while the amino acid residue at position 44 is isoleucine. Optionally, the amino acid residue at position 43 may then be alanine or glutamic acid.

In some embodiments, the amino acid residues at the positions corresponding to positions 1, 2, 3 and 4 or to positions 3, 4, 5 and 6 in SEQ ID NO: 4-7 have been deleted. In specific variants of these embodiments, the parental polypeptide is the C domain of Protein A (SEQ ID NO: 5). The effects of these deletions on the native C domain are described in U.S. Pat. Nos. 9,018,305 and 8,329,860, which are hereby incorporated by reference in their entireties.

In certain embodiments, the mutation in SEQ ID NO 4-7, such as in SEQ ID NO 7, is selected from the group consisting of:
N11K; N11E; N11Y; N11T; N11F; N11L; N11W; N11I; N11M; N11V; N11A; N11H; N1IR; N11E,Q32A; N11E,Q32E,Q40E; N11E,Q32E,K50R; Q9A,N11E,N43A; Q9A,N11E,N28A,N43A; Q9A,N11E,Q40V,A42K,N43E,L44I; Q9A,N11E,Q40V,A42K,N43A,L44I; N11K,H18K,S33K,D37E,A42R,N43A,L44I,K50R,L51Y; Q9A,N11E,N28A,Q40V,A42K,N43A,L44I; Q9A,N11K,H18K,S33K,D37E,A42R,N43A,L44I,K50R,L51Y; N11K, H18K, D37E, A42R, N43A, L44I; Q9A, N11K, H18K, D37E, A42R, N43A, L44I; Q9A, N11K, H18K, D37E, A42R, N43A, L44I, K50R; Q9A,N11K,H18K,D37E,A42R; Q9A,N11E,D37E,Q40V,A42K,N43A,L44I and Q9A,N11E,D37E,Q40V,A42R,N43A,L44I.

These mutations provide particularly high alkaline stabilities. The mutation in SEQ ID NO 4-7, such as in SEQ ID NO 7, can also be selected from the group consisting of
N11K; N11Y; N11F; N11L; N11W; N11I; N11M; N11V; N11A; N11H; N11R; Q9A,N11E,N43A; Q9A,N11E,N28A,N43A; Q9A,N11E,Q40V,A42K,N43E,L44I; Q9A,N11E,Q40V,A42K,N43A,L44I; Q9A,N11E,N28A,Q40V,A42K,N43A,L44I; N11K,H18K,S33K,D37E,A42R,N43A,L44I,K50R,L51Y; Q9A,N11K,H18K,S33K,D37E,A42R,N43A,L44I,K50R,L51Y; N11K, H18K, D37E, A42R, N43A, L44I; Q9A, N11K, H18K, D37E, A42R, N43A, L44I and Q9A, N11K, H18K, D37E, A42R, N43A, L44I, K50R.

In some embodiments, the polypeptide comprises or consists essentially of a sequence selected from the group consisting of:
SEQ ID NO 8, SEQ ID NO 9, SEQ ID NO 10, SEQ ID NO 11, SEQ ID NO 12, SEQ ID NO 13, SEQ ID NO 14, SEQ ID NO 15, SEQ ID NO 16, SEQ ID NO 23, SEQ ID NO 24, SEQ ID NO 25, SEQ ID NO 26, SEQ ID NO 27, SEQ ID NO 28, SEQ ID NO 29, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49 and SEQ ID NO 50.

It may e.g. comprise or consist essentially of a sequence selected from the group consisting of:
SEQ ID NO 8, SEQ ID NO 9, SEQ ID NO 10, SEQ ID NO 11, SEQ ID NO 16, SEQ ID NO 23, SEQ ID NO 24, SEQ ID NO 25, SEQ ID NO 26, SEQ ID NO 27, SEQ ID NO 28 and SEQ ID NO 29.

It can also comprise or consist essentially of a sequence selected from the group consisting of:
SEQ ID NO 8, SEQ ID NO 9, SEQ ID NO 10, SEQ ID NO 11, SEQ ID NO 16, SEQ ID NO 23, SEQ ID NO 24, SEQ ID NO 25, SEQ ID NO 27, SEQ ID NO 28, SEQ ID NO 38, SEQ ID NO 40; SEQ ID NO 41; SEQ ID NO 42; SEQ NO 43, SEQ ID NO 44, SEQ ID NO 45, SEQ ID NO 46, SEQ ID NO 47 and SEQ ID NO 48.

The polypeptide may e.g. be defined by a sequence selected from the groups above or from subsets of these groups, but it may also comprise additional amino acid residues at the N- and/or C-terminal end, e.g. a leader sequence at the N-terminal end and/or a tail sequence at the C-terminal end.

Zvar(Q9A, N11E, N43A)
SEQ ID NO 8
VDAKFDKEAQ EAFYEILHLP NLTEEQRNAF IQSLKDDPSQ SAALLAEAKK
LNDAQAPK
Zvar(Q9A, N11E, N28A, N43A)
SEQ ID NO 9
VDAKFDKEAQ EAFYEILHLP NLTEEQRAAF IQSLKDDPSQ SAALLAEAKK
LNDAQAPK
Zvar(Q9A, N11E, Q40V, A42K, N43E, L44I)
SEQ ID NO 10
VDAKFDKEAQ EAFYEILHLP NLTEEQRNAF IQSLKDDPSV SKEILAEAKK
LNDAQAPK
Zvar(Q9A, N11E, Q40V, A42K, N43A, L44I)
SEQ ID NO 11
VDAKFDKEAQ EAFYEILHLP NLTEEQRNAF IQSLKDDPSV SKAILAEAKK
LNDAQAPK
Zvar(N11E, Q32A)
SEQ ID NO 12
VDAKFDKEQQ EAFYEILHLP NLTEEQRNAF IASLKDDPSQ SANLLAEAKK
LNDAQAPK
Zvar(N11E)
SEQ ID NO 13
VDAKFDKEQQ EAFYEILHLP NLTEEQRNAF IQSLKDDPSQ SANLLAEAKK
LNDAQAPK
Zvar(N11E, Q32E, Q40E)
SEQ ID NO 14
VDAKFDKEQQ EAFYEILHLP NLTEEQRNAF IESLKDDPSE SANLLAEAKK
LNDAQAPK
Zvar(N11E, Q32E, K50R)
SEQ ID NO 15
VDAKFDKEQQ EAFYEILHLP NLTEEQRNAF IESLKDDPSQ SANLLAEAKR
LNDAQAPK
Zvar(N11K)
SEQ ID NO 16
VDAKFDKEQQ KAFYEILHLP NLTEEQRNAF IQSLKDDPSQ SANLLAEAKK
LNDAQAPK
Zvar(N11K, H18K, S33K, D37E, A42R, N43A, L44I, K50R, L51Y)
SEQ ID NO 23
VDAKFDKEQQ KAFYEILKLP NLTEEQRNAF IQKLKDEPSQ SRAILAEAKR
YNDAQAPK
Zvar(Q9A, N11E, N28A, Q40V, A42K, N43A, L44I)
SEQ ID NO 24
VDAKFDKEAQ EAFYEILHLP NLTEEQRAAF IQSLKDDPSV SKAILAEAKK
LNDAQAPK
Zvar(Q9A, N11K, H18K, S33K, D37E, A42R, N43A, L44I, K50R, L51Y)
SEQ ID NO 25
VDAKFDKEAQ KAFYEILKLP NLTEEQRAAF IQKLKDEPSQ SRAILAEAKR
YNDAQAPK
Zvar(N11K, H18K, D37E, A42R, N43A, L44I)
SEQ ID NO 26
VDAKFDKEQQ KAFYEILKLP NLTEEQRNAF IQSLKDEPSQ SRAILAEAKK
LNDAQAPK
Zvar(Q9A, N11K, H18K, D37E, A42R, N43A, L44I)
SEQ ID NO 27
VDAKFDKEAQ KAFYEILKLP NLTEEQRNAF IQSLKDEPSQ SRAILAEAKK
LNDAQAPK
Zvar(Q9A, N11K, H18K, D37E, A42R, N43A, L44I, K50R)
SEQ ID NO 28
VDAKFDKEAQ KAFYEILKLP NLTEEQRNAF IQSLKDEPSQ SRAILAEAKR
LNDAQAPK
Zvar(Q9A, N11K, H18K, D37E, A42R)
SEQ ID NO 29
VDAKFDKEAQ KAFYEILKLP NLTEEQRNAF IQSLKDEPSQ SRNLLAEAKK
LNDAQAPK
B(Q9A, N11E, Q40V, A42K, N43A, L44I)
SEQ ID NO 36
ADNKFNKEAQ EAFYEILHLP NLNEEQRNGF IQSLKDDPSV SKAILAEAKK
LNDAQAPK
C(Q9A, N11E, E43A)
SEQ ID NO 37
ADNKFNKEAQ EAFYEILHLP NLTEEQRNGF IQSLKDDPSV SKAILAEAKK
LNDAQAPK
Zvar(N11Y)
SEQ ID NO 38
VDAKFDKEQQ YAFYEILHLP NLTEEQRNAF IQSLKDDPSQ SANLLAEAKK
LNDAQAPK
Zvar(N11T)
SEQ ID NO 39
VDAKFDKEQQ TAFYEILHLP NLTEEQRNAF IQSLKDDPSQ SANLLAEAKK
LNDAQAPK
Zvar(N11F)
SEQ ID NO 40
VDAKFDKEQQ FAFYEILHLP NLTEEQRNAF IQSLKDDPSQ SANLLAEAKK
LNDAQAPK
Zvar(N11L)
SEQ ID NO 41
VDAKFDKEQQ LAFYEILHLP NLTEEQRNAF IQSLKDDPSQ SANLLAEAKK
LNDAQAPK
Zvar(N11W)
SEQ ID NO 42
VDAKFDKEQQ WAFYEILHLP NLTEEQRNAF IQSLKDDPSQ SANLLAEAKK
LNDAQAPK
Zvar(N11I)
SEQ ID NO 43
VDAKFDKEQQ IAFYEILHLP NLTEEQRNAF IQSLKDDPSQ SANLLAEAKK
LNDAQAPK
Zvar(N11M)
SEQ ID NO 44
VDAKFDKEQQ MAFYEILHLP NLTEEQRNAF IQSLKDDPSQ SANLLAEAKK
LNDAQAPK
Zvar(N11V)
SEQ ID NO 45
VDAKFDKEQQ VAFYEILHLP NLTEEQRNAF IQSLKDDPSQ SANLLAEAKK
LNDAQAPK
Zvar(N11A)
SEQ ID NO 46
VDAKFDKEQQ AAFYEILHLP NLTEEQRNAF IQSLKDDPSQ SANLLAEAKK
LNDAQAPK
Zvar(N11H)
SEQ ID NO 47
VDAKFDKEQQ HAFYEILHLP NLTEEQRNAF IQSLKDDPSQ SANLLAEAKK
LNDAQAPK
Zvar(N11R)
SEQ ID NO 48
VDAKFDKEQQ RAFYEILHLP NLTEEQRNAF IQSLKDDPSQ SANLLAEAKK
LNDAQAPK
Zvar(Q9A, N11E, D37E, Q40V, A42K, N43A, L44I)
SEQ ID NO 49
VDAKFDKEAQ EAFYEILHLP NLTEEQRNAF IQSLKDEPSV SKAILAEAKK
LNDAQAPK
Zvar(Q9A, N11E, D37E, Q40V, A42R, N43A, L44I)
SEQ ID NO 50
VDAKFDKEAQ EAFYEILHLP NLTEEQRNAF IQSLKDEPSV SRAILAEAKK
LNDAQAPK

In a second aspect the present invention discloses a multimer comprising, or consisting essentially of, a plurality of polypeptide units as defined by any embodiment disclosed above. The multimer can e.g. be a dimer, a trimer, a tetramer, a pentamer, a hexamer, a heptamer, an octamer or a nonamer. It can be a homomultimer, where all the units in the multimer are identical or it can be a heteromultimer, where at least one unit differs from the others. Advantageously, all the units in the multimer are alkali stable, such as by comprising the mutations disclosed above. The polypeptides can be linked to each other directly by peptide bonds between the C-terminal and N-terminal ends of the polypeptides. Alternatively, two or more units in the multimer can be linked by linkers comprising oligomeric or polymeric species, such as elements comprising up to 15 or 30 amino acids, such as 1-5, 1-10 or 5-10 amino acids. This is the case in particular for mutations of SEQ ID NO 51 and 52 and for the SEQ ID NO 53 polypeptide, where specific examples of linkers can e.g. be VDAKFD or ADNKFN, such as VDAKFD. The nature of such a linker should preferably not destabilize the spatial conformation of the protein units. This can e.g. be achieved by avoiding the presence of proline in the linkers. Furthermore, said linker should preferably also be sufficiently stable in alkaline environments not to impair the properties of the mutated protein units. For this purpose, it is advantageous if the linkers do not contain asparagine. It can additionally be advantageous if the linkers do not contain glutamine. The multimer may further at the N-terminal end comprise a plurality of amino acid residues e.g. originating from the cloning process or constituting a residue from a cleaved off signaling sequence. The number of additional amino acid residues may e.g. be 15 or less, such as 10 or less or 5 or less. As a specific example, the multimer may comprise an AQ sequence at the N-terminal end.

In certain embodiments, the multimer may comprise, or consist essentially, of a sequence selected from the group consisting of: SEQ ID NO 17, SEQ ID NO 18, SEQ ID NO 19, SEQ ID NO 20, SEQ ID NO 30, SEQ ID NO 31, SEQ ID NO 32, SEQ ID NO 33, SEQ ID NO 34 and SEQ ID NO 35. These sequences are listed below and named as Parent (Mutations)n, where n is the number of monomer units in a multimer.

Zvar(Q9A, N11E, N43A)4
SEQ ID NO 17
AQGT VDAKFDKEAQ EAFYEILHLP NLTEEQRNAF IQSLKDDPSQ SAALLAEAKK
LNDAQAPK VDAKFDKEAQ EAFYEILHLP NLTEEQRNAF IQSLKDDPSQ
SAALLAEAKK LNDAQAPK VDAKFDKEAQ EAFYEILHLP NLTEEQRNAF
IQSLKDDPSQ SAALLAEAKK LNDAQAPK VDAKFDKEAQ EAFYEILHLP
NLTEEQRNAF IQSLKDDPSQ SAALLAEAKK LNDAQAPKC
Zvar(Q9A, N11E, N28A, N43A)4
SEQ ID NO 18
AQGT VDAKFDKEAQ EAFYEILHLP NLTEEQRAAF IQSLKDDPSQ SAALLAEAKK
LNDAQAPK VDAKFDKEAQ EAFYEILHLP NLTEEQRAAF IQSLKDDPSQ
SAALLAEAKK LNDAQAPK VDAKFDKEAQ EAFYEILHLP NLTEEQRAAF
IQSLKDDPSQ SAALLAEAKK LNDAQAPK VDAKFDKEAQ EAFYEILHLP
NLTEEQRAAF IQSLKDDPSQ SAALLAEAKK LNDAQAPKC
Zvar(Q9A, N11E, Q40V, A42K, N43E, L44I)4
SEQ ID NO 19
AQGT VDAKFDKEAQ EAFYEILHLP NLTEEQRNAF IQSLKDDPSV SKEILAEAKK
LNDAQAPK VDAKFDKEAQ EAFYEILHLP NLTEEQRNAF IQSLKDDPSV
SKEILAEAKK LNDAQAPK VDAKFDKEAQ EAFYEILHLP NLTEEQRNAF
IQSLKDDPSV SKEILAEAKK LNDAQAPK VDAKFDKEAQ EAFYEILHLP
NLTEEQRNAF IQSLKDDPSV SKEILAEAKK LNDAQAPKC
Zvar(Q9A, N11E, Q40V, A42K, N43A, L44I)4
SEQ ID NO 20
AQGT VDAKFDKEAQ EAFYEILHLP NLTEEQRNAF IQSLKDDPSV SKAILAEAKK
LNDAQAPK VDAKFDKEAQ EAFYEILHLP NLTEEQRNAF IQSLKDDPSV
SKAILAEAKK LNDAQAPK VDAKFDKEAQ EAFYEILHLP NLTEEQRNAF
IQSLKDDPSV SKAILAEAKK LNDAQAPK VDAKFDKEAQ EAFYEILHLP
NLTEEQRNAF IQSLKDDPSV SKAILAEAKK LNDAQAPKC
Zvar(N11K, H18K, S33K, D37E, A42R, N43A, L44I, K50R, L51Y)4
SEQ ID NO 30
AQGT VDAKFDKEQQ KAFYEILKLP NLTEEQRNAF IQKLKDEPSQ SRAILAEAKR
YNDAQAPK VDAKFDKEQQ KAFYEILKLP NLTEEQRNAF IQKLKDEPSQ
SRAILAEAKR YNDAQAPK VDAKFDKEQQ KAFYEILKLP NLTEEQRNAF
IQKLKDEPSQ SRAILAEAKR YNDAQAPK VDAKFDKEQQ KAFYEILKLP
NLTEEQRNAF IQKLKDEPSQ SRAILAEAKR YNDAQAPKC
Zvar(Q9A, N11K, H18K, D37E, A42R)4
SEQ ID NO 31
AQGT VDAKFDKEAQ KAFYEILKLP NLTEEQRNAF IQSLKDEPSQ SRNLLAEAKK
LNDAQAPK VDAKFDKEAQ KAFYEILKLP NLTEEQRNAF IQSLKDEPSQ
SRNLLAEAKK LNDAQAPK VDAKFDKEAQ KAFYEILKLP NLTEEQRNAF
IQSLKDEPSQ SRNLLAEAKK LNDAQAPK VDAKFDKEAQ KAFYEILKLP
NLTEEQRNAF IQSLKDEPSQ SRNLLAEAKK LNDAQAPKC
Zvar(Q9A, N11E, N28A, Q40V, A42K, N43A, L44I)4
SEQ ID NO 32
AQGT VDAKFDKEAQ EAFYEILHLP NLTEEQRAAF IQSLKDDPSV SKAILAEAKK
LNDAQAPK VDAKFDKEAQ EAFYEILHLP NLTEEQRAAF IQSLKDDPSV
SKAILAEAKK LNDAQAPK VDAKFDKEAQ EAFYEILHLP NLTEEQRAAF
IQSLKDDPSV SKAILAEAKK LNDAQAPK VDAKFDKEAQ EAFYEILHLP
NLTEEQRAAF IQSLKDDPSV SKAILAEAKK LNDAQAPKC
Zvar(Q9A, N11E, Q40V, A42K, N43A, L44I)6
SEQ ID NO 33
AQGT VDAKFDKEAQ EAFYEILHLP NLTEEQRNAF IQSLKDDPSV SKAILAEAKK
LNDAQAPK VDAKFDKEAQ EAFYEILHLP NLTEEQRNAF IQSLKDDPSV
SKAILAEAKK LNDAQAPK VDAKFDKEAQ EAFYEILHLP NLTEEQRNAF
IQSLKDDPSV SKAILAEAKK LNDAQAPK VDAKFDKEAQ EAFYEILHLP
NLTEEQRNAF IQSLKDDPSV SKAILAEAKK LNDAQAPK VDAKFDKEAQ
EAFYEILHLP NLTEEQRNAF IQSLKDDPSV SKAILAEAKK LNDAQAPK
VDAKFDKEAQ EAFYEILHLP NLTEEQRNAF IQSLKDDPSV SKAILAEAKK
LNDAQAPKC
Zvar(Q9A, N11E, D37E, Q40V, A42K, N43A, L44I)4
SEQ ID NO 34
AQGT VDAKFDKEAQ EAFYEILHLP NLTEEQRNAF IQSLKDEPSV SKAILAEAKK
LNDAQAPK VDAKFDKEAQ EAFYEILHLP NLTEEQRNAF IQSLKDEPSV
SKAILAEAKK LNDAQAPK VDAKFDKEAQ EAFYEILHLP NLTEEQRNAF
IQSLKDEPSV SKAILAEAKK LNDAQAPK VDAKFDKEAQ EAFYEILHLP
NLTEEQRNAF IQSLKDEPSV SKAILAEAKK LNDAQAPKC
Zvar(Q9A, N11E, D37E, Q40V, A42R, N43A, L44I)4
SEQ ID NO 35
AQGT VDAKFDKEAQ EAFYEILHLP NLTEEQRNAF IQSLKDEPSV SRAILAEAKK
LNDAQAPK VDAKFDKEAQ EAFYEILHLP NLTEEQRNAF IQSLKDEPSV
SRAILAEAKK LNDAQAPK VDAKFDKEAQ EAFYEILHLP NLTEEQRNAF
IQSLKDEPSV SRAILAEAKK LNDAQAPK VDAKFDKEAQ EAFYEILHLP
NLTEEQRNAF IQSLKDEPSV SRAILAEAKK LNDAQAPKC

In some embodiments, the polypeptide and/or multimer, as disclosed above, further comprises at the C-terminal or N-terminal end one or more coupling elements, selected from the group consisting of one or more cysteine residues, a plurality of lysine residues and a plurality of histidine residues. The coupling element(s) may also be located within 1-5 amino acid residues, such as within 1-3 or 1-2 amino acid residues from the C-terminal or N-terminal end. The coupling element may e.g. be a single cysteine at the C-terminal end. The coupling element(s) may be directly linked to the C- or N-terminal end, or it/they may be linked via a stretch comprising up to 15 amino acids, such as 1-5, 1-10 or 5-10 amino acids. This stretch should preferably also be sufficiently stable in alkaline environments not to impair the properties of the mutated protein. For this purpose, it is advantageous if the stretch does not contain asparagine. It can additionally be advantageous if the stretch does not contain glutamine. An advantage of having a C-terminal cysteine is that endpoint coupling of the protein can be achieved through reaction of the cysteine thiol with an electrophilic group on a support. This provides excellent mobility of the coupled protein which is important for the binding capacity.

The alkali stability of the polypeptide or multimer can be assessed by coupling it to an SPR chip, e.g. to Biacore CM5 sensor chips as described in the examples, using e.g. NHS- or maleimide coupling chemistries, and measuring the immunoglobulin-binding capacity of the chip, typically using polyclonal human IgG, before and after incubation in alkaline solutions at a specified temperature, e.g. 22+/−2° C. The incubation can e.g. be performed in 0.5 M NaOH for a number of 10 min cycles, such as 100, 200 or 300 cycles. The IgG capacity of the matrix after 100 10 min incubation cycles in 0.5 M NaOH at 22+/−2° C. can be at least 55, such as at least 60, at least 80 or at least 90% of the IgG capacity before the incubation. Alternatively, the remaining IgG capacity after 100 cycles for a particular mutant measured as above can be compared with the remaining IgG capacity for the parental polypeptide/multimer. In this case, the remaining IgG capacity for the mutant may be at least 105%, such as at least 110%, at least 125%, at least 150% or at least 200% of the parental polypeptide/multimer.

In a third aspect the present invention discloses a nucleic acid encoding a polypeptide or multimer according to any embodiment disclosed above. Thus, the invention encompasses all forms of the present nucleic acid sequence such as the RNA and the DNA encoding the polypeptide or multimer. The invention embraces a vector, such as a plasmid, which in addition to the coding sequence comprises the required signal sequences for expression of the polypeptide or multimer according the invention. In one embodiment, the vector comprises nucleic acid encoding a multimer according to the invention, wherein the separate nucleic acids encoding each unit may have homologous or heterologous DNA sequences.

In a fourth aspect the present invention discloses an expression system, which comprises, a nucleic acid or a vector as disclosed above. The expression system may e.g. be a gram-positive or gram-negative prokaryotic host cell system, e.g. E. coli or Bacillus sp. which has been modified to express the present polypeptide or multimer. In an alternative embodiment, the expression system is a eukaryotic host cell system, such as a yeast, e.g. Pichia pastoris or Saccharomyces cerevisiae, or mammalian cells, e.g. CHO cells.

In a fifth aspect, the present invention discloses a separation matrix, wherein a plurality of polypeptides or multimers according to any embodiment disclosed above have been coupled to a solid support. Such a matrix is useful for separation of immunoglobulins or other Fc-containing proteins and, due to the improved alkali stability of the polypeptides/multimers, the matrix will withstand highly alkaline conditions during cleaning, which is essential for long-term repeated use in a bioprocess separation setting. The alkali stability of the matrix can be assessed by measuring the immunoglobulin-binding capacity, typically using polyclonal human IgG, before and after incubation in alkaline solutions at a specified temperature, e.g. 22+/−2° C. The incubation can e.g. be performed in 0.5 M or 1.0 M NaOH for a number of 15 min cycles, such as 100, 200 or 300 cycles, corresponding to a total incubation time of 25, 50 or 75 h. The IgG capacity of the matrix after 96-100 15 min incubation cycles or a total incubation time of 24 or 25 h in 0.5 M NaOH at 22+/−2° C. can be at least 80, such as at least 85, at least 90 or at least 95% of the IgG capacity before the incubation. The capacity of the matrix after a total incubation time of 24 h in 1.0 M NaOH at 22+/−2° C. can be at least 70, such as at least 80 or at least 90% of the IgG capacity before the incubation.

As the skilled person will understand, the expressed polypeptide or multimer should be purified to an appropriate extent before being immobilized to a support. Such purification methods are well known in the field, and the immobilization of protein-based ligands to supports is easily carried out using standard methods. Suitable methods and supports will be discussed below in more detail.

The solid support of the matrix according to the invention can be of any suitable well-known kind. A conventional affinity separation matrix is often of organic nature and based on polymers that expose a hydrophilic surface to the aqueous media used, i.e. expose hydroxy (—OH), carboxy (—COOH), carboxamido (—CONH₂, possibly in N-substituted forms), amino (—NH₂, possibly in substituted form), oligo- or polyethylenoxy groups on their external and, if present, also on internal surfaces. The solid support can suitably be porous. The porosity can be expressed as a Kav or Kd value (the fraction of the pore volume available to a probe molecule of a particular size) measured by inverse size exclusion chromatography, e.g. according to the methods described in Gel Filtration Principles and Methods, Pharmacia LKB Biotechnology 1991, pp 6-13. By definition, both Kd and Kav values always lie within the range 0-1. The Kav value can advantageously be 0.6-0.95, e.g. 0.7-0.90 or 0.6-0.8, as measured with dextran of Mw 110 kDa as a probe molecule. An advantage of this is that the support has a large fraction of pores able to accommodate both the polypeptides/multimers of the invention and immunoglobulins binding to the polypeptides/multimers and to provide mass transport of the immunoglobulins to and from the binding sites.

The polypeptides or multimers may be attached to the support via conventional coupling techniques utilising e.g. thiol, amino and/or carboxy groups present in the ligand. Bisepoxides, epichlorohydrin, CNBr, N-hydroxysuccinimide (NHS) etc are well-known coupling reagents. Between the support and the polypeptide/multimer, a molecule known as a spacer can be introduced, which improves the availability of the polypeptide/multimer and facilitates the chemical coupling of the polypeptide/multimer to the support. Depending on the nature of the polypeptide/multimer and the coupling conditions, the coupling may be a multipoint coupling (e.g. via a plurality of lysines) or a single point coupling (e.g. via a single cysteine). Alternatively, the polypeptide/multimer may be attached to the support by non-covalent bonding, such as physical adsorption or biospecific adsorption.

In some embodiments the matrix comprises 5-25, such as 5-20 mg/ml, 5-15 mg/ml, 5-11 mg/ml or 6-11 mg/ml of the polypeptide or multimer coupled to the support. The amount of coupled polypeptide/multimer can be controlled by the concentration of polypeptide/multimer used in the coupling process, by the activation and coupling conditions used and/or by the pore structure of the support used. As a general rule the absolute binding capacity of the matrix increases with the amount of coupled polypeptide/multimer, at least up to a point where the pores become significantly constricted by the coupled polypeptide/multimer. The relative binding capacity per mg coupled polypeptide/multimer will decrease at high coupling levels, resulting in a cost-benefit optimum within the ranges specified above.

In certain embodiments the polypeptides or multimers are coupled to the support via thioether bonds. Methods for performing such coupling are well-known in this field and easily performed by the skilled person in this field using standard techniques and equipment. Thioether bonds are flexible and stable and generally suited for use in affinity chromatography. In particular when the thioether bond is via a terminal or near-terminal cysteine residue on the polypeptide or multimer, the mobility of the coupled polypeptide/multimer is enhanced which provides improved binding capacity and binding kinetics. In some embodiments the polypeptide/multimer is coupled via a C-terminal cysteine provided on the protein as described above. This allows for efficient coupling of the cysteine thiol to electrophilic groups, e.g. epoxide groups, halohydrin groups etc. on a support, resulting in a thioether bridge coupling.

In certain embodiments the support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups and they are generally stable towards alkaline cleaning solutions used in bioprocessing.

In some embodiments the support comprises agar or agarose. The supports used in the present invention can easily be prepared according to standard methods, such as inverse suspension gelation (S Hjertén: Biochim Biophys Acta 79 (2), 393-398 (1964). Alternatively, the base matrices are commercially available products, such as crosslinked agarose beads sold under the name of SEPHAROSE™ FF (GE Healthcare). In an embodiment, which is especially advantageous for large-scale separations, the support has been adapted to increase its rigidity using the methods described in U.S. Pat. No. 6,602,990 or U.S. Pat. No. 7,396,467, which are hereby incorporated by reference in their entirety, and hence renders the matrix more suitable for high flow rates.

In certain embodiments the support, such as a polysaccharide or agarose support, is crosslinked, such as with hydroxyalkyl ether crosslinks. Crosslinker reagents producing such crosslinks can be e.g. epihalohydrins like epichlorohydrin, diepoxides like butanediol diglycidyl ether, allylating reagents like allyl halides or allyl glycidyl ether. Crosslinking is beneficial for the rigidity of the support and improves the chemical stability. Hydroxyalkyl ether crosslinks are alkali stable and do not cause significant nonspecific adsorption.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L′Industria 70 (9), 70-75 (1988)). Alternatively, a commercially available product, such as SOURCE™ (GE Healthcare) is used. In another alternative, the solid support according to the invention comprises a support of inorganic nature, e.g. silica, zirconium oxide etc.

In yet another embodiment, the solid support is in another form such as a surface, a chip, capillaries, or a filter (e.g. a membrane or a depth filter matrix).

As regards the shape of the matrix according to the invention, in one embodiment the matrix is in the form of a porous monolith. In an alternative embodiment, the matrix is in beaded or particle form that can be porous or non-porous. Matrices in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

In a sixth aspect, the present invention discloses a method of isolating an immunoglobulin, wherein a separation matrix as disclosed above is used.

In certain embodiments, the method comprises the steps of:
a) contacting a liquid sample comprising an immunoglobulin with a separation matrix as disclosed above,
b) washing said separation matrix with a washing liquid,
c) eluting the immunoglobulin from the separation matrix with an elution liquid, and
d) cleaning the separation matrix with a cleaning liquid, which can alternatively be called a cleaning-in-place (CIP) liquid, e.g. with a contact (incubation) time of at least 10 min.

The method may also comprise steps of, before step a), providing an affinity separation matrix according to any of the embodiments described above and providing a solution comprising an immunoglobulin and at least one other substance as a liquid sample and of, after step c), recovering the eluate and optionally subjecting the eluate to further separation steps, e.g. by anion or cation exchange chromatography, multimodal chromatography and/or hydrophobic interaction chromatography. Suitable compositions of the liquid sample, the washing liquid and the elution liquid, as well as the general conditions for performing the separation are well known in the art of affinity chromatography and in particular in the art of Protein A chromatography. The liquid sample comprising an Fc-containing protein and at least one other substance may comprise host cell proteins (HCP), such as CHO cell, E Coli or yeast proteins. Contents of CHO cell and E Coli proteins can conveniently be determined by immunoassays directed towards these proteins, e.g. the CHO HCP or E Coli HCP ELISA kits from Cygnus Technologies. The host cell proteins or CHO cell/E Coli proteins may be desorbed during step b).

The elution may be performed by using any suitable solution used for elution from Protein A media. This can e.g. be a solution or buffer with pH 5 or lower, such as pH 2.5-5 or 3-5. It can also in some cases be a solution or buffer with pH 11 or higher, such as pH 11-14 or pH 11-13. In some embodiments the elution buffer or the elution buffer gradient comprises at least one mono-di- or trifunctional carboxylic acid or salt of such a carboxylic acid. In certain embodiments the elution buffer or the elution buffer gradient comprises at least one anion species selected from the group consisting of acetate, citrate, glycine, succinate, phosphate, and formiate.

In some embodiments, the cleaning liquid is alkaline, such as with a pH of 13-14. Such solutions provide efficient cleaning of the matrix, in particular at the upper end of the interval

In certain embodiments, the cleaning liquid comprises 0.1-2.0 M NaOH or KOH, such as 0.5-2.0 or 0.5-1.0 M NaOH or KOH. These are efficient cleaning solutions, and in particular so when the NaOH or KOH concentration is above 0.1 M or at least 0.5 M. The high stability of the polypeptides of the invention enables the use of such strongly alkaline solutions.

The method may also include a step of sanitizing the matrix with a sanitization liquid, which may e.g. comprise a peroxide, such as hydrogen peroxide and/or a peracid, such as peracetic acid or performic acid.

In some embodiments, steps a)-d) are repeated at least 10 times, such as at least 50 times, 50-200, 50-300 or 50-500 times. This is important for the process economy in that the matrix can be re-used many times.

Steps a)-c) can also be repeated at least 10 times, such as at least 50 times, 50-200, 50-300 or 50-500 times, with step d) being performed after a plurality of instances of step c), such that step d) is performed at least 10 times, such as at least 50 times. Step d) can e.g. be performed every second to twentieth instance of step c).

### EXAMPLES

### Mutagenesis of Protein

Site-directed mutagenesis was performed by a two-step PCR using oligonucleotides coding for the mutations. As template a plasmid containing a single domain of either Z, B or C was used. The PCR fragments were ligated into an E. coli expression vector. DNA sequencing was used to verify the correct sequence of inserted fragments. To form multimers of mutants an Acc I site located in the starting codons (GTA GAC) of the B, C or Z domain was used, corresponding to amino acids VD. The vector for the monomeric domain was digested with Acc I and phosphatase treated. Acc I sticky-ends primers were designed, specific for each variant, and two overlapping PCR products were generated from each template. The PCR products were purified and the concentration was estimated by comparing the PCR products on a 2% agarose gel. Equal amounts of the pair wise PCR products were hybridized (90° C.→25° C. in 45 min) in ligation buffer. The resulting product consists approximately to ¼ of fragments likely to be ligated into an Acc I site (correct PCR fragments and/or the digested vector). After ligation and transformation colonies were PCR screened to identify constructs containing the desired mutant. Positive clones were verified by DNA sequencing.

### Construct Expression and Purification

The constructs were expressed in the bacterial periplasm by fermentation of E. coli K12 in standard media. After fermentation the cells were heat-treated to release the periplasm content into the media. The constructs released into the medium were recovered by microfiltration with a membrane having a 0.2 μm pore size.

Each construct, now in the permeate from the filtration step, was purified by affinity. The permeate was loaded onto a chromatography medium containing immobilized IgG (IgG Sepharose 6FF, GE Healthcare). The loaded product was washed with phosphate buffered saline and eluted by lowering the pH.

The elution pool was adjusted to a neutral pH (pH 8) and reduced by addition of dithiothreitol. The sample was then loaded onto an anion exchanger. After a wash step the construct was eluted in a NaCl gradient to separate it from any contaminants. The elution pool was concentrated by ultrafiltration to 40-50 mg/ml. It should be noted that the successful affinity purification of a construct on an immobilized IgG medium indicates that the construct in question has a high affinity to IgG.

The purified ligands were analyzed with RPC LC-MS to determine the purity and to ascertain that the molecular weight corresponded to the expected (based on the amino acid sequence).

### Example 1

The purified monomeric ligands listed in Table 1, further comprising an AQGT leader sequence at the N-terminus and a cysteine at the C terminus, were immobilized on Biacore CM5 sensor chips (GE Healthcare, Sweden), using the amine coupling kit of GE Healthcare (for carbodiimide coupling of amines on the carboxymethyl groups on the chip) in an amount sufficient to give a signal strength of about 200-1500 RU in a Biacore surface plasmon resonance (SPR) instrument (GE Healthcare, Sweden). To follow the IgG binding capacity of the immobilized surface 1 mg/ml human polyclonal IgG (Gammanorm) was flowed over the chip and the signal strength (proportional to the amount of binding) was noted. The surface was then cleaned-in-place (CIP), i.e. flushed with 500 mM NaOH for 10 minutes at room temperature (22+/−2° C.). This was repeated for 96-100 cycles and the immobilized ligand alkaline stability was followed as the remaining IgG binding capacity (signal strength) after each cycle. The results are shown in Table 1 and indicate that at least the ligands Zvar (N11K)1, Zvar (N11E)1, Zvar (N11Y)1, Zvar (N11T)1, Zvar (N11F)1, Zvar (N11L)1, Zvar (N11W)1, ZN11I)1, Zvar (N11M)1, Zvar (N11V)1, Zvar (N11A)1, Zvar (N11H1), Zvar (N11R)1, Zvar (N11E,Q32A)1, Zvar (N11E,Q32E,Q40E)1 and Zvar (N11E,Q32E,K50R)1, Zvar (Q9A,N11E,N43A)1, Zvar (Q9A,N11E,N28A,N43A)1, Zvar (Q9A,N11E,Q40V,A42K,N43E,L44I)1, Zvar (Q9A,N11E,Q40V,A42K,N43A,L44I)1, Zvar (Q9A,N11E,N28A,Q40V,A42K,N43A,L44I)1, Zvar (N11K,H18K,S33K,D37E,A42R,N43A,L44I,K50R,L51Y)1, Zvar (Q9A,N11K,H18K,S33K,D37E,A42R,N43A,L44I,K50R,L51Y)1, Zvar (N11K, H18K, D37E, A42R, N43A, L441)1, Zvar (Q9A, N11K, H18K, D37E, A42R, N43A, L44I)1 and Zvar (Q9A, N11K, H18K, D37E, A42R, N43A, L44I, K50R)1 have an improved alkali stability compared to the parental structure Zvar1, used as the reference. Further, the ligands B (Q9A,N11E,Q40V,A42K,N43A,L44I)1 and C (Q9A,N11E,E43A)1 have an improved stability compared to the parental B and C domains, used as references.

TABLE 1
Monomeric ligands, evaluated by Biacore (0.5M NaOH).

| | | | Reference | |
|---|---|---|---|---|
| | | Capacity | capacity | Capacity |
| | | after 96- | after 96- | relative to |
| Ligand | Sequence | 100 cycles | 100 cycles | reference |
| Zvar(N11E, Q32A)1 | SEQ ID | 57% | 55% | 1.036 |
| | NO 12 | | | |
| Zvar(N11E)1 | SEQ ID | 59% | 55% | 1.073 |
| | NO 13 | | | |
| Zvar(N11E, Q32E, | SEQ ID | 52% | 51% | 1.020 |
| Q40E)1 | NO 14 | | | |
| Zvar(N11E, Q32E, | SEQ ID | 53% | 51% | 1.039 |
| K50R)1 | NO 15 | | | |
| Zvar(N11K)1 | SEQ ID | 62% | 49% | 1.270 |
| | NO 16 | | | |
| Zvar(N11Y)1 | SEQ ID | 55% | 46% | 1.20 |
| | NO 38 | | | |
| Zvar(N11T)1 | SEQ ID | 50% | 46% | 1.09 |
| | NO 39 | | | |
| Zvar(N11F)1 | SEQ ID | 55% | 46% | 1.20 |
| | NO 40 | | | |
| Zvar(N11L)1 | SEQ ID | 57% | 47% | 1.21 |
| | NO 41 | | | |
| Zvar(N11W)1 | SEQ ID | 57% | 47% | 1.21 |
| | NO 42 | | | |
| Zvar(N11I)1 | SEQ ID | 57% | 47% | 1.21 |
| | NO 43 | | | |
| Zvar(N11M)1 | SEQ ID | 58% | 46% | 1.26 |
| | NO 44 | | | |
| Zvar(N11V)1 | SEQ ID | 56% | 46% | 1.22 |
| | NO 45 | | | |
| Zvar(N11A)1 | SEQ ID | 58% | 46% | 1.26 |
| | NO 46 | | | |
| Zvar(N11H)1 | SEQ ID | 57% | 46% | 1.24 |
| | NO 47 | | | |
| Zvar(N11R)1 | SEQ ID | 59% | 46% | 1.28 |
| | NO 48 | | | |
| Zvar(Q9A, N11E, | SEQ ID | 70% | 47% | 1.49 |
| N43A)1 | NO 8 | | | |
| Zvar(Q9A, N11E, N28A, | SEQ ID | 68% | 47% | 1.45 |
| N43A)1 | NO 9 | | | |
| Zvar(Q9A, N11E, Q40V, | SEQ ID | 67% | 47% | 1.43 |
| A42K, N43E, L44I)1 | NO 10 | | | |
| Zvar(Q9A, N11E, Q40V, | SEQ ID | 66% | 47% | 1.40 |
| A42K, N43A, L44I)1 | NO 11 | | | |
| Zvar(Q9A, N11E, N28A, | SEQ ID | 65% | 48% | 1.35 |
| Q40V, A42K, N43A, | NO 24 | | | |
| L44I)1 | | | | |
| Zvar(N11K, H18K, | SEQ ID | 67% | 46% | 1.46 |
| S33K, D37E, A42R, | NO 23 | | | |
| N43A, L44I, K50R, | | | | |
| L51Y)1 | | | | |
| Zvar(Q9A, N11K, | SEQ ID | 59% | 46% | 1.28 |
| H18K, S33K, D37E, | NO 25 | | | |
| A42R, N43A, L44I, | | | | |
| K50R, L51Y)1 | | | | |
| Zvar(N11K, H18K, | SEQ ID | 59% | 45% | 1.31 |
| D37E, A42R, N43A, | NO 26 | | | |
| L44I)1 | | | | |
| Zvar(Q9A, N11K, | SEQ ID | 63% | 45% | 1.40 |
| H18K, D37E, A42R, | NO 27 | | | |
| N43A, L44I)1 | | | | |
| Zvar(Q9A, N11K, | SEQ ID | 67% | 45% | 1.49 |
| H18K, D37E, A42R, | NO 28 | | | |
| N43A, L44I, K50R)1 | | | | |
| B(Q9A, N11E, Q40V, | SEQ ID | 39% | 35% | 1.11 |
| A42K, N43A, L44I)1 | NO 36 | | | |
| C(Q9A, N11E, E43A)1 | SEQ ID | 60% | 49% | 1.22 |
| | NO 37 | | | |

### Example 2

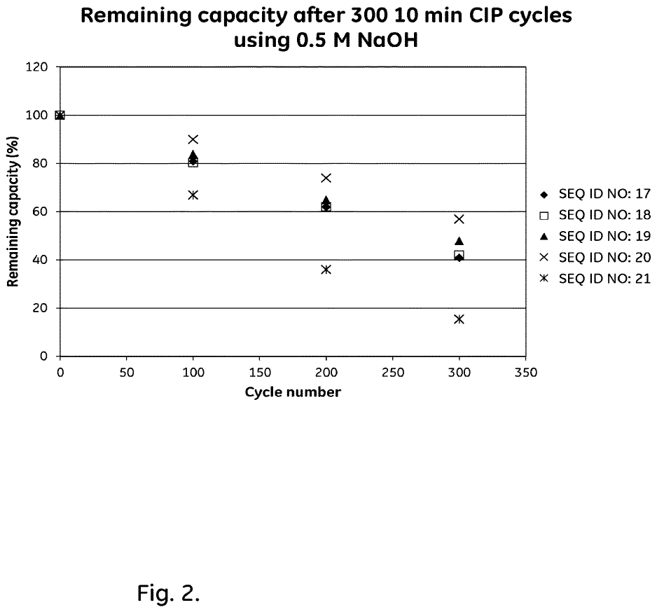

The purified tetrameric and hexameric ligands listed in Table 2 were immobilized on Biacore CM5 sensor chips (GE Healthcare, Sweden), using the amine coupling kit of GE Healthcare (for carbodiimide coupling of amines on the carboxymethyl groups on the chip) in an amount sufficient to give a signal strength of about 200-1500 RU in a Biacore instrument (GE Healthcare, Sweden). To follow the IgG binding capacity of the immobilized surface 1 mg/ml human polyclonal IgG (Gammanorm) was flowed over the chip and the signal strength (proportional to the amount of binding) was noted. The surface was then cleaned-in-place (CIP), i.e. flushed with 500 mM NaOH for 10 minutes at room temperature (22+/−2° C.). This was repeated for 300 cycles and the immobilized ligand alkaline stability was followed as the remaining IgG binding capacity (signal strength) after each cycle. The results are shown in Table 2 and in FIG. 2 and indicate that at least the ligands Zvar (Q9A,N11E,N43A)4, Zvar (Q9A,N11E,N28A,N43A)4, Zvar (Q9A,N11E,Q40V,A42K,N43E,L441)4 and Zvar (Q9A,N11E,Q40V,A42K,N43A,L441)4, Zvar (Q9A,N11E,D37E,Q40V,A42K,N43A,L441)4 and Zvar (Q9A,N11E,D37E,Q40V,A42R,N43A,L44I)4 have an improved alkali stability compared to the parental structure Zvar4, which was used as a reference. The hexameric ligand Zvar (Q9A,N11E,Q40V,A42K,N43A,L441)6 also has improved alkali stability compared to the parental structure Zvar6, used as a reference.

TABLE 2
Tetrameric and hexameric ligands, evaluated by Biacore (0.5M NaOH).

| | | Remaining | Capacity | Remaining | Capacity | Remaining | Capacity |
|---|---|---|---|---|---|---|---|
| | SEQ | capacity | relative | capacity | relative | capacity | relative |
| | ID | 100 cycles | to ref. | 200 cycles | to ref. | 300 cycles | to ref. |
| Ligand | NO: | (%) | 100 cycles | (%) | 200 cycles | (%) | 300 cycles |
| Zvar4 | 21 | 67 | 1 | 36 | 1 | 16 | 1 |
| Zvar(Q9A, N11E, N43A)4 | 17 | 81 | 1.21 | 62 | 1.72 | 41 | 2.56 |
| Zvar(Q9A, N11E, N28A, | 18 | 80 | 1.19 | 62 | 1.72 | 42 | 2.62 |
| N43A)4 | | | | | | | |
| Zvar(Q9A, N11E, Q40V, | 19 | 84 | 1.25 | 65 | 1.81 | 48 | 3.00 |
| A42K, N43E, L44I)4 | | | | | | | |
| Zvar(Q9A, N11E, Q40V, | 20 | 90 | 1.34 | 74 | 2.06 | 57 | 3.56 |
| A42K, N43A, L44I)4 | | | | | | | |
| Zvar(Q9A, N11E, N28A, | 32 | 84 | 1.24 | Not tested | Not tested | Not tested | Not tested |
| Q40V, A42K, N43A, L44I)4 | | | | | | | |
| Zvar(Q9A, N11E, Q40V, | 33 | 87 | 1.30 | Not tested | Not tested | Not tested | Not tested |
| A42K, N43A, L44I)6 | | | | | | | |
| Zvar(Q9A, N11E, D37E, | 34 | 81 | 1.13 | Not tested | Not tested | Not tested | Not tested |
| Q40V, A42K, N43A, L44I)4 | | | | | | | |
| Zvar(Q9A, N11E, D37E, | 35 | 84 | 1.17 | Not tested | Not tested | Not tested | Not tested |
| Q40V, A42R, N43A, L44I)4 | | | | | | | |

### Example 3

Example 2 was repeated with 100 CIP cycles of three ligands using 1 M NaOH instead of 500 mM as in Example 2. The results are shown in Table 3 and show that all three ligands have an improved alkali stability also in 1M NaOH, compared to the parental structure Zvar4 which was used as a reference.

TABLE 3
Tetrameric ligands, evaluated by Biacore (1M NaOH).

| | | | Capacity |
|---|---|---|---|
| | | Remaining | relative to |
| | | capacity 100 | ref. 100 |
| Ligand | Sequence | cycles (%) | cycles |
| Zvar4 | SEQ ID | 27 | 1 |
| | NO 21 | | |
| Zvar(Q9A, N11E, N28A, N43A)4 | SEQ ID | 55 | 2.04 |
| | NO 18 | | |
| Zvar(Q9A, N11E, Q40V, A42K, | SEQ ID | 54 | 2.00 |
| N43E, L44I)4 | NO 19 | | |
| Zvar(Q9A, N11E, Q40V, A42K, | SEQ ID | 56 | 2.07 |
| N43A, L44I)4 | NO 20 | | |

### Example 4

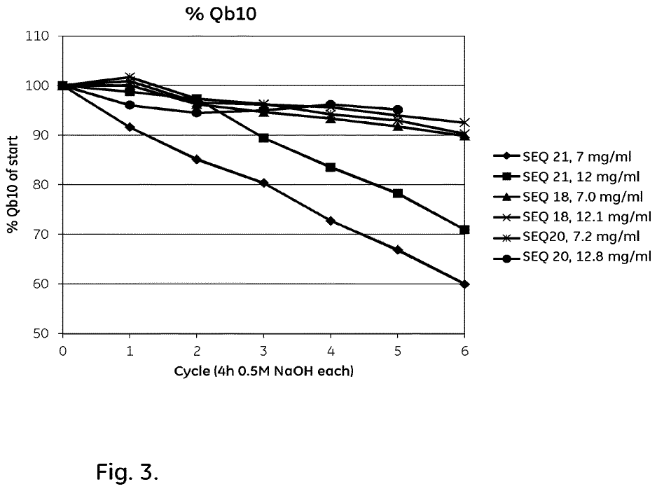

The purified tetrameric ligands of Table 2 (all with an additional N-terminal cysteine) were immobilized on agarose beads using the methods described below and assessed for capacity and stability. The results are shown in Table 4 and FIG. 3.

TABLE 4
Matrices with tetrametric ligands, evaluated in columns (0.5M NaOH).

| | | | | Remaining | | |
|---|---|---|---|---|---|---|
| | | | | IgG | Remaining | Capacity |
| | | | Initial | capacity | IgG | retention |
| | | | IgG | Qb10 | capacity | relative |
| | SEQ | Ligand | capacity | after six | after six | to ref. |
| | ID | content | Qb10 | 4 h cycles | 4 h cycles | after six |
| | NO. | (mg/ml) | (mg/ml) | (mg/ml) | (%) | 4 h cycles |
| Zvar4 | 21 | 7 | 52.5 | 36.5 | 60 | 1 |
| Zvar4 | 21 | 12 | 61.1 | 43.4 | 71 | 1 |
| Zvar(Q9A, N11E, N28A, N43A)4 | 18 | 7.0 | 49.1 | 44.1 | 90 | 1.50 |
| Zvar(Q9A, N11E, N28A, N43A)4 | 18 | 12.1 | 50.0 | 46.2 | 93 | 1.31 |
| Zvar(Q9A, N11E, Q40V, A42K, N43A, L44I)4 | 20 | 7.2 | 49.0 | 44.2 | 90 | 1.50 |
| Zvar(Q9A, N11E, Q40V, A42K, N43A, L44I)4 | 20 | 12.8 | 56.3 | 53.6 | 95 | 1.34 |
| Zvar(N11K, H18K, S33K, D37E, A42R, N43A, | 30 | 9.7 | 56.3 | 52.0 | 92 | 1.53 |
| L44I, K50R, L51Y)4 | | | | | | |
| Zvar(Q9A, N11K, H18K, D37E, A42R)4 | 31 | 10.8 | 56.9 | 52.5 | 92 | 1.30 |

### Activation

The base matrix used was rigid cross-linked agarose beads of 85 micrometers (volume-weighted, d50V) median diameter, prepared according to the methods of U.S. Pat. No. 6,602,990 and with a pore size corresponding to an inverse gel filtration chromatography Kav value of 0.70 for dextran of Mw 110 kDa, according to the methods described in Gel Filtration Principles and Methods, Pharmacia LKB Biotechnology 1991, pp 6-13.

25 mL (g) of drained base matrix, 10.0 mL distilled water and 2.02 g NaOH(s) was mixed in a 100 mL flask with mechanical stirring for 10 min at 25° C. 4.0 mL of epichlorohydrin was added and the reaction progressed for 2 hours. The activated gel was washed with 10 gel sediment volumes (GV) of water.

### Coupling

To 20 mL of ligand solution (50 mg/mL) in a 50 ml Falcon tube, 169 mg NaHCO₃, 21 mg Na₂CO₃, 175 mg NaCl and 7 mg EDTA, was added. The Falcon tube was placed on a roller table for 5-10 min, and then 77 mg of DTE was added. Reduction proceeded for >45 min. The ligand solution was then desalted on a PD10 column packed with Sephadex G-25. The ligand content in the desalted solution was determined by measuring the 276 nm UV absorption.

The activated gel was washed with 3-5 GV {0.1 M phosphate/1 mM EDTA pH 8.6} and the ligand was then coupled according to the method described in U.S. Pat. No. 6,399,750. All buffers used in the experiments had been degassed by nitrogen gas for at least 5-10 min. The ligand content of the gels could be controlled by varying the amount and concentration of the ligand solution.

After immobilization the gels were washed 3×GV with distilled water. The gels+1 GV {0.1 M phosphate/1 mM EDTA/10% thioglycerol pH 8.6} was mixed and the tubes were left in a shaking table at room temperature overnight. The gels were then washed alternately with 3×GV {0.1 M TRIS/0.15 M NaCl pH 8.6} and 0.5 M HAc and then 8-10×GV with distilled water. Gel samples were sent to an external laboratory for amino acid analysis and the ligand content (mg/ml gel) was calculated from the total amino acid content.

### Protein

Gammanorm 165 mg/ml (Octapharma), diluted to 2 mg/ml in Equilibration buffer.

### Equilibration Buffer

PBS Phosphate buffer 10 mM+0.14 M NaCl+0.0027 M KCl, pH 7.4 (Medicago)

### Adsorption Buffer

PBS Phosphate buffer 10 mM+0.14 M NaCl+0.0027 M KCl, pH 7.4 (Medicago)

### Elution Buffers

100 mM acetate pH 2.9

### Dynamic Binding Capacity

2 ml of resin was packed in TRICORN™ 5 100 columns. The breakthrough capacity was determined with an ÄKTAExplorer 10 system at a residence time of 6 minutes (0.33 ml/min flow rate). Equilibration buffer was run through the bypass column until a stable baseline was obtained. This was done prior to auto zeroing. Sample was applied to the column until a 100% UV signal was obtained. Then, equilibration buffer was applied again until a stable baseline was obtained.

Sample was loaded onto the column until a UV signal of 85% of maximum absorbance was reached. The column was then washed with 5 column volumes (CV) equilibration buffer at flow rate 0.5 ml/min. The protein was eluted with 5 CV elution buffer at a flow rate of 0.5 ml/min. Then the column was cleaned with 0.5M NaOH at flow rate 0.2 ml/min and re-equilibrated with equilibration buffer.

For calculation of breakthrough capacity at 10%, the equation below was used. That is the amount of IgG that is loaded onto the column until the concentration of IgG in the column effluent is 10% of the IgG concentration in the feed.

$${q}_{10 \%} = \frac{{C}_{0}}{{V}_{c}} [ {V}_{app} - {V}_{sys} - {\int }_{{V}_{sys}}^{{V}_{app}} \frac{A ( V ) - {A}_{sub}}{{A}_{100 \%} - {A}_{sub}} ⋆ d v ]$$

A_{100%}=100% UV signal;
A_{sub}=absorbance contribution from non-binding IgG subclass;
A(V)=absorbance at a given applied volume;
V_{c}=column volume;
Vₐₚₚ=volume applied until 10% breakthrough;
V_{sys}=system dead volume;
C₀=feed concentration.

The dynamic binding capacity (DBC) at 10% breakthrough was calculated. The dynamic binding capacity (DBC) was calculated for 10 and 80% breakthrough.

### CIP-0.5 M NaOH

The 10% breakthrough DBC (Qb10) was determined both before and after repeated exposures to alkaline cleaning solutions. Each cycle included a CIP step with 0.5 M NaOH pumped through the column at a rate of 0.5/min for 20 min, after which the column was left standing for 4 h. The exposure took place at room temperature (22+/−2° C.). After this incubation, the column was washed with equilibration buffer for 20 min at a flow rate of 0.5 ml/min. Table 4 shows the remaining capacity after six 4 h cycles (i.e. 24 h cumulative exposure time to 0.5 M NaOH), both in absolute numbers and relative to the initial capacity.

### Example 5

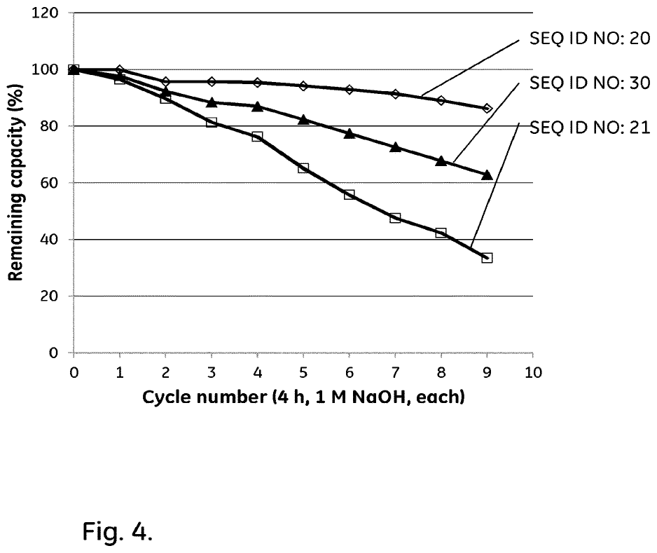

Example 4 was repeated with the tetrameric ligands shown in Table 5, but with 1.0 M NaOH used in the CIP steps instead of 0.5 M. The results are shown in Table 5 and in FIG. 4.

TABLE 5
Matrices with tetrametric ligands, evaluated in columns - 1.0M NaOH.

| | | | | Remaining | | |
|---|---|---|---|---|---|---|
| | | | | IgG | Remaining | Capacity |
| | | | Initial | capacity | IgG | retention |
| | | | IgG | Qb10 | capacity | relative |
| | SEQ | Ligand | capacity | after six | after six | to ref. |
| | ID | content | Qb10 | 4 h cycles | 4 h cycles | after six |
| | NO. | (mg/ml) | (mg/ml) | (mg/ml) | (%) | 4 h cycles |
| Zvar4 | 21 | 12 | 60.1 | 33.5 | 56 | 1 |
| Zvar(Q9A, N11E, Q40V, A42K, N43A, L44I)4 | 20 | 12.8 | 60.3 | 56.0 | 93 | 1.67 |
| Zvar(N11K, H18K, S33K, D37E, A42R, N43A, | 30 | 9.7 | 62.1 | 48.1 | 77 | 1.44 |
| LA4I, K50R, L51Y)4 | | | | | | |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

1-49. (canceled)

50. A polypeptide capable of binding to an Fc region of an antibody, wherein the polypeptide comprises at least one monomeric unit comprising the amino acid sequence of SEQ ID NO: 11.

51. A polypeptide capable of binding to an Fc region of an antibody, wherein the polypeptide comprises four monomeric units, each monomeric unit comprising the amino acid sequence of SEQ ID NO:11.

52. The polypeptide of claim 51, wherein the monomeric units are directly linked by peptide bonds between the C-terminus of one monomeric unit and the N-terminus of the adjacent monomeric unit.

53. The polypeptide of claim 51, wherein the polypeptide comprises a leader sequence at the N-terminal end and/or a tail sequence at the C-terminal end.

54. The polypeptide of claim 51, wherein the polypeptide comprises the amino acid sequence of SEQ ID NO: 20.

55. A nucleic acid or a vector encoding the polypeptide of claim 50.

56. A nucleic acid or a vector encoding the polypeptide of claim 54.

57. An expression system comprising the nucleic acid or vector of claim 55.

58. An expression system comprising the nucleic acid or vector of claim 56.

59. A separation matrix comprising at least one polypeptide of claim 50 coupled to a solid support.

60. The separation matrix of claim 59, wherein the at least one polypeptide is coupled to the solid support via thiol, amino and/or carboxy groups.

61. The separation matrix of claim 60, wherein the at least one polypeptide is coupled to the support via thioether bonds.

62. The separation matrix of claim 59, wherein the multimer retains at least about 70% IgG binding capacity after 100 cycles of exposure to 0.5 M NaOH.

63. The separation matrix of claim 59, wherein the multimer retains at least about 80% IgG binding capacity after 24 hours of exposure to 0.5 M NaOH.

64. A separation matrix comprising at least one polypeptide of claim 54 coupled to a solid support.

65. The separation matrix of claim 64, wherein the at least one polypeptide is coupled to the solid support via thiol, amino and/or carboxy groups.

66. The separation matrix of claim 65, wherein the at least one polypeptide is coupled to the support via thioether bonds.

67. The separation matrix of claim 64, wherein the polypeptide retains at least about 70% IgG binding capacity after 100 cycles of exposure to 0.5 M NaOH.

68. The separation matrix of claim 64, wherein the polypeptide retains at least about 80% IgG binding capacity after 24 hours of exposure to 0.5 M NaOH.


SEQUENCE LISTING (53 sequences)

SEQ ID NO: 1 - protein, 51 aa, Staphylococcus aureus
AQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 58 aa, Escherichia coli [sequence omitted]

SEQ ID NO: 7 - protein, 58 aa, Escherichia coli [sequence omitted]

SEQ ID NO: 8 - protein, 58 aa, Escherichia coli
VDAKFDKEAQEAFYEILHLPNLTEEQRNAFIQSLKDDPSQSAALLAEAKKLNDAQAPK

SEQ ID NO: 9 - protein, 58 aa, Escherichia coli
VDAKFDKEAQEAFYEILHLPNLTEEQRAAFIQSLKDDPSQSAALLAEAKKLNDAQAPK

SEQ ID NO: 10 - protein, 58 aa, Escherichia coli
VDAKFDKEAQEAFYEILHLPNLTEEQRNAFIQSLKDDPSVSKEILAEAKKLNDAQAPK

SEQ ID NO: 11 - protein, 58 aa, Escherichia coli
VDAKFDKEAQEAFYEILHLPNLTEEQRNAFIQSLKDDPSVSKAILAEAKKLNDAQAPK

SEQ ID NO: 12 - protein, 58 aa, Escherichia coli
VDAKFDKEQQEAFYEILHLPNLTEEQRNAFIASLKDDPSQSANLLAEAKKLNDAQAPK

SEQ ID NO: 13 - protein, 58 aa, Escherichia coli
VDAKFDKEQQEAFYEILHLPNLTEEQRNAFIQSLKDDPSQSANLLAEAKKLNDAQAPK

SEQ ID NO: 14 - protein, 58 aa, Escherichia coli
VDAKFDKEQQEAFYEILHLPNLTEEQRNAFIESLKDDPSESANLLAEAKKLNDAQAPK

SEQ ID NO: 15 - protein, 58 aa, Escherichia coli
VDAKFDKEQQEAFYEILHLPNLTEEQRNAFIESLKDDPSQSANLLAEAKRLNDAQAPK

SEQ ID NO: 16 - protein, 58 aa, Escherichia coli
VDAKFDKEQQKAFYEILHLPNLTEEQRNAFIQSLKDDPSQSANLLAEAKKLNDAQAPK

SEQ ID NO: 17 - protein, 237 aa, Escherichia coli
AQGTVDAKFDKEAQEAFYEILHLPNLTEEQRNAFIQSLKDDPSQSAALLAEAKKLNDAQAPKVDAKFDKEAQEAFYEILHLPNLTEEQRNAFIQSLKDDPSQSAALLAEAKKLNDAQAPKVDAKFDKEAQEAFYEILHLPNLTEEQRNAFIQSLKDDPSQSAALLAEAKKLNDAQAPKVDAKFDKEAQEAFYEILHLPNLTEEQRNAFIQSLKDDPSQSAALLAEAKKLNDAQAPKC

SEQ ID NO: 18 - protein, 237 aa, Escherichia coli
AQGTVDAKFDKEAQEAFYEILHLPNLTEEQRAAFIQSLKDDPSQSAALLAEAKKLNDAQAPKVDAKFDKEAQEAFYEILHLPNLTEEQRAAFIQSLKDDPSQSAALLAEAKKLNDAQAPKVDAKFDKEAQEAFYEILHLPNLTEEQRAAFIQSLKDDPSQSAALLAEAKKLNDAQAPKVDAKFDKEAQEAFYEILHLPNLTEEQRAAFIQSLKDDPSQSAALLAEAKKLNDAQAPKC

SEQ ID NO: 19 - protein, 237 aa, Escherichia coli
AQGTVDAKFDKEAQEAFYEILHLPNLTEEQRNAFIQSLKDDPSVSKEILAEAKKLNDAQAPKVDAKFDKEAQEAFYEILHLPNLTEEQRNAFIQSLKDDPSVSKEILAEAKKLNDAQAPKVDAKFDKEAQEAFYEILHLPNLTEEQRNAFIQSLKDDPSVSKEILAEAKKLNDAQAPKVDAKFDKEAQEAFYEILHLPNLTEEQRNAFIQSLKDDPSVSKEILAEAKKLNDAQAPKC

SEQ ID NO: 20 - protein, 237 aa, Escherichia coli
AQGTVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKC

SEQ ID NO: 21 - protein, 237 aa, Escherichia coli
AQGTVDAKFDKEQQNAFYEILHLPNLTEEQRNAFIQSLKDDPSQSANLLAEAKKLNDAQAPKVDAKFDKEQQNAFYEILHLPNLTEEQRNAFIQSLKDDPSQSANLLAEAKKLNDAQAPKVDAKFDKEQQNAFYEILHLPNLTEEQRNAFIQSLKDDPSQSANLLAEAKKLNDAQAPKVDAKFDKEQQNAFYEILHLPNLTEEQRNAFIQSLKDDPSQSANLLAEAKKLNDAQAPKC

SEQ ID NO: 22 - protein, 58 aa, Escherichia coli
ADNNFNKEQQNAFYEILNMPNLNEEQRNGFIQSLKDDPSQSANLLSEAKKLNESQAPK

SEQ ID NO: 23 - protein, 58 aa, Escherichia coli
VDAKFDKEQQKAFYEILKLPNLTEEQRNAFIQKLKDEPSQSRAILAEAKRYNDAQAPK

SEQ ID NO: 24 - protein, 58 aa, Escherichia coli
VDAKFDKEAQEAFYEILHLPNLTEEQRAAFIQSLKDDPSVSKAILAEAKKLNDAQAPK

SEQ ID NO: 25 - protein, 58 aa, Escherichia coli
VDAKFDKEAQKAFYEILKLPNLTEEQRAAFIQKLKDEPSQSRAILAEAKRYNDAQAPK

SEQ ID NO: 26 - protein, 58 aa, Escherichia coli
VDAKFDKEQQKAFYEILKLPNLTEEQRNAFIQSLKDEPSQSRAILAEAKKLNDAQAPK

SEQ ID NO: 27 - protein, 58 aa, Escherichia coli
VDAKFDKEAQKAFYEILKLPNLTEEQRNAFIQSLKDEPSQSRAILAEAKKLNDAQAPK

SEQ ID NO: 28 - protein, 58 aa, Escherichia coli
VDAKFDKEAQKAFYEILKLPNLTEEQRNAFIQSLKDEPSQSRAILAEAKRLNDAQAPK

SEQ ID NO: 29 - protein, 58 aa, Escherichia coli
VDAKFDKEAQKAFYEILKLPNLTEEQRNAFIQSLKDEPSQSRNLLAEAKKLNDAQAPK

SEQ ID NO: 30 - protein, 237 aa, Escherichia coli
AQGTVDAKFDKEQQKAFYEILKLPNLTEEQRNAFIQKLKDEPSQSRAILAEAKRYNDAQAPKVDAKFDKEQQKAFYEILKLPNLTEEQRNAFIQKLKDEPSQSRAILAEAKRYNDAQAPKVDAKFDKEQQKAFYEILKLPNLTEEQRNAFIQKLKDEPSQSRAILAEAKRYNDAQAPKVDAKFDKEQQKAFYEILKLPNLTEEQRNAFIQKLKDEPSQSRAILAEAKRYNDAQAPKC

SEQ ID NO: 31 - protein, 237 aa, Escherichia coli
AQGTVDAKFDKEAQKAFYEILKLPNLTEEQRNAFIQSLKDEPSQSRNLLAEAKKLNDAQAPKVDAKFDKEAQKAFYEILKLPNLTEEQRNAFIQSLKDEPSQSRNLLAEAKKLNDAQAPKVDAKFDKEAQKAFYEILKLPNLTEEQRNAFIQSLKDEPSQSRNLLAEAKKLNDAQAPKVDAKFDKEAQKAFYEILKLPNLTEEQRNAFIQSLKDEPSQSRNLLAEAKKLNDAQAPKC

SEQ ID NO: 32 - protein, 237 aa, Escherichia coli
AQGTVDAKFDKEAQEAFYEILHLPNLTEEQRAAFIQSLKDDPSVSKAILAEAKKLNDAQAPKVDAKFDKEAQEAFYEILHLPNLTEEQRAAFIQSLKDDPSVSKAILAEAKKLNDAQAPKVDAKFDKEAQEAFYEILHLPNLTEEQRAAFIQSLKDDPSVSKAILAEAKKLNDAQAPKVDAKFDKEAQEAFYEILHLPNLTEEQRAAFIQSLKDDPSVSKAILAEAKKLNDAQAPKC

SEQ ID NO: 33 - protein, 353 aa, Escherichia coli
AQGTVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKVDAKFDKEAQEAFYEILHLPNLTEEQRNAFIQSLKDDPSVSKAILAEAKKLNDAQAPKC

SEQ ID NO: 34 - protein, 237 aa, Escherichia coli
AQGTVDAKFDKEAQEAFYEILHLPNLTEEQRNAFIQSLKDEPSVSKAILAEAKKLNDAQAPKVDAKFDKEAQEAFYEILHLPNLTEEQRNAFIQSLKDEPSVSKAILAEAKKLNDAQAPKVDAKFDKEAQEAFYEILHLPNLTEEQRNAFIQSLKDEPSVSKAILAEAKKLNDAQAPKVDAKFDKEAQEAFYEILHLPNLTEEQRNAFIQSLKDEPSVSKAILAEAKKLNDAQAPKC

SEQ ID NO: 35 - protein, 237 aa, Escherichia coli
AQGTVDAKFDKEAQEAFYEILHLPNLTEEQRNAFIQSLKDEPSVSRAILAEAKKLNDAQAPKVDAKFDKEAQEAFYEILHLPNLTEEQRNAFIQSLKDEPSVSRAILAEAKKLNDAQAPKVDAKFDKEAQEAFYEILHLPNLTEEQRNAFIQSLKDEPSVSRAILAEAKKLNDAQAPKVDAKFDKEAQEAFYEILHLPNLTEEQRNAFIQSLKDEPSVSRAILAEAKKLNDAQAPKC

SEQ ID NO: 36 - protein, 58 aa, Escherichia coli
ADNKFNKEAQEAFYEILHLPNLNEEQRNGFIQSLKDDPSVSKAILAEAKKLNDAQAPK

SEQ ID NO: 37 - protein, 58 aa, Escherichia coli
ADNKFNKEAQEAFYEILHLPNLTEEQRNGFIQSLKDDPSVSKAILAEAKKLNDAQAPK

SEQ ID NO: 38 - protein, 58 aa, Escherichia coli
VDAKFDKEQQYAFYEILHLPNLTEEQRNAFIQSLKDDPSQSANLLAEAKKLNDAQAPK

SEQ ID NO: 39 - protein, 58 aa, Escherichia coli
VDAKFDKEQQTAFYEILHLPNLTEEQRNAFIQSLKDDPSQSANLLAEAKKLNDAQAPK

SEQ ID NO: 40 - protein, 58 aa, Escherichia coli
VDAKFDKEQQFAFYEILHLPNLTEEQRNAFIQSLKDDPSQSANLLAEAKKLNDAQAPK

SEQ ID NO: 41 - protein, 58 aa, Escherichia coli
VDAKFDKEQQLAFYEILHLPNLTEEQRNAFIQSLKDDPSQSANLLAEAKKLNDAQAPK

SEQ ID NO: 42 - protein, 58 aa, Escherichia coli
VDAKFDKEQQWAFYEILHLPNLTEEQRNAFIQSLKDDPSQSANLLAEAKKLNDAQAPK

SEQ ID NO: 43 - protein, 58 aa, Escherichia coli
VDAKFDKEQQIAFYEILHLPNLTEEQRNAFIQSLKDDPSQSANLLAEAKKLNDAQAPK

SEQ ID NO: 44 - protein, 58 aa, Escherichia coli
VDAKFDKEQQMAFYEILHLPNLTEEQRNAFIQSLKDDPSQSANLLAEAKKLNDAQAPK

SEQ ID NO: 45 - protein, 58 aa, Escherichia coli
VDAKFDKEQQAAFYEILHLPNLTEEQRNAFIQSLKDDPSQSANLLAEAKKLNDAQAPK

SEQ ID NO: 46 - protein, 58 aa, Escherichia coli
VDAKFDKEQQHAFYEILHLPNLTEEQRNAFIQSLKDDPSQSANLLAEAKKLNDAQAPK

SEQ ID NO: 47 - protein, 58 aa, Escherichia coli
VDAKFDKEQQRAFYEILHLPNLTEEQRNAFIQSLKDDPSQSANLLAEAKKLNDAQAPK

SEQ ID NO: 48 - protein, 58 aa, Escherichia coli
VDAKFDKEAQEAFYEILHLPNLTEEQRNAFIQSLKDEPSVSKAILAEAKKLNDAQAPK

SEQ ID NO: 49 - protein, 58 aa, Escherichia coli; identical to SEQ ID NO: 48

SEQ ID NO: 50 - protein, 58 aa, Escherichia coli
VDAKFDKEAQEAFYEILHLPNLTEEQRNAFIQSLKDEPSVSRAILAEAKKLNDAQAPK

SEQ ID NO: 51 - protein, 52 aa, Escherichia coli
KEQQNAFYEILHLPNLTEEQRNAFIQSLKDDPSQSANLLAEAKKLNDAQAPK

SEQ ID NO: 52 - protein, 52 aa, Escherichia coli
KEQQNAFYEILHLPNLTEEQRNGFIQSLKDDPSVSKEILAEAKKLNDAQAPK

SEQ ID NO: 53 - protein, 52 aa, Escherichia coli
  VARIANT 3: note = X can be any naturally occurring amino acid
  VARIANT 5: note = X can be any naturally occurring amino acid
  VARIANT 12: note = X can be any naturally occurring amino acid
  VARIANT 22: note = X can be any naturally occurring amino acid
  VARIANT 23: note = X can be any naturally occurring amino acid
  VARIANT 26: note = X can be any naturally occurring amino acid
  VARIANT 27: note = X can be any naturally occurring amino acid
  VARIANT 31: note = X can be any naturally occurring amino acid
  VARIANT 34: note = X can be any naturally occurring amino acid
  VARIANT 36: note = X can be any naturally occurring amino acid
  VARIANT 37: note = X can be any naturally occurring amino acid
  VARIANT 38: note = X can be any naturally occurring amino acid
  VARIANT 44: note = X can be any naturally occurring amino acid
  VARIANT 45: note = X can be any naturally occurring amino acid
KEXQXAFYEILXLPNLTEEQRXXFIXXLKDXPSXSXXXLAEAKXXNDAQAPK